(19) United States
(12) Patent Application Publication
(10) Pub. No.: US 2023/0302178 A1
(43) Pub. Date: Sep. 28, 2023

(54) METHOD OF REMOVING PATHOGENS FROM A SURFACE

(71) Applicant: 3M INNOVATIVE PROPERTIES COMPANY, St. Paul, MN (US)

(72) Inventors: Andrew W. Vail, Bayport, MN (US); Robert A. Schmitz, St. Paul, MN (US)

(21) Appl. No.: 18/023,112

(22) PCT Filed: Aug. 26, 2021
(86) PCT No.: PCT/IB2021/057833

§ 371 Date: Feb. 24, 2023

Related U.S. Application Data
(60) Provisional application No. 63/072,761, filed on Aug. 31, 2020.

(51) Int. Cl. A61L 2/18

(52) U.S. Cl. CPC A61L 2/18; A61L 2101/18

(57) ABSTRACT

A method of removing pathogens from a surface is described. In particular, a method including providing a wipe including guanidinyl-containing polymer coating, providing a liquid disinfectant having an associated disinfection time t for at least one pathogen is described. The method may help provide safe levels of disinfection even with a significant associated error in dwell time.

### BACKGROUND

Removing pathogens from a surface is an important step in cleaning and or disinfecting a space. The removal of pathogens is essential to limiting the spread of transmissible diseases and infections. Especially with self-replicating pathogens (for example, bacteria), insufficient removal may lead to a reestablishment of infectious levels of disease-causing agents.

### SUMMARY

In one aspect, the present disclosure relates to a method of removing pathogens from a surface. The method includes providing a wipe including a guanidinyl-containing polymer coating, providing a liquid disinfectant having an associated disinfection time for at least one pathogen; contacting a surface with the wipe carrying the liquid disinfectant; allowing the liquid disinfectant to be in contact with the surface for a time, t, until removal or evaporation. t is the associated disinfection time for the at least one pathogen, with an associated error. The associated error is ±40% of the associated disinfection time for the at least one pathogen.

### BRIEF DESCRIPTION OF THE DRAWINGS

FIG. 1 is a graph showing the relationship between disinfection and dwell time for both an embodiment of a method of removing pathogens from a surface described herein and for a conventional method.

FIG. 2 is a top perspective schematic of contacting a surface with a wipe carrying a liquid disinfectant.

### DETAILED DESCRIPTION

Removing pathogens from a surface is an important step in many settings for reducing 30 the chance of disease transmission from infectious agents. In contexts such as a hospital or other healthcare facility, the level and frequency of disinfection may be specified by government regulation. A specified disinfection threshold may also be required for compliance with internal procedures. Of course, a robust and effective disinfection program can also improve patient outcomes and reduce costs for a healthcare facility by minimizing hospital-acquired infections.

For example, a particular disinfectant may have a certain disinfection time associated with it. Commercial disinfectants typically must remain in contact with the surface for a certain time (the dwell time) in order to reach the stated disinfection level. In many situations, it may be difficult to actually keep the disinfectant in contact with the surface long enough to meet this “kill claim.” Vertical walls or surfaces like doorknobs and table legs may be difficult to keep wet for long enough. And, if a sufficient quantity is not applied, the disinfectant may evaporate before having a sufficiently long dwell time.

In practice, well-intentioned disinfection regimens will include a significant error between intended dwell time and actual dwell time. Surfaces in a room will likely not be treated simultaneously and the timing may be difficult to keep track of. Liquid disinfectant may be inadvertently wiped up before the dwell time is reached. Interruptions or distractions may lead to mistakes.

This error may significantly increase the risk of an unacceptable level of residual pathogens. A failed inspection may result in fines or other penalties or the rate of hospital-acquired infections may increase.

The method described herein of utilizing a wipe including a guanidinyl-containing polymer coating may provide extra safeguards against unintentionally insufficient disinfection. The method described herein may allow for errors in dwell times of up to 40% while still providing disinfection levels consistent with or exceeding that of a conventional method with perfect, error-free (e.g., testing lab environment) compliance with a stated dwell time.

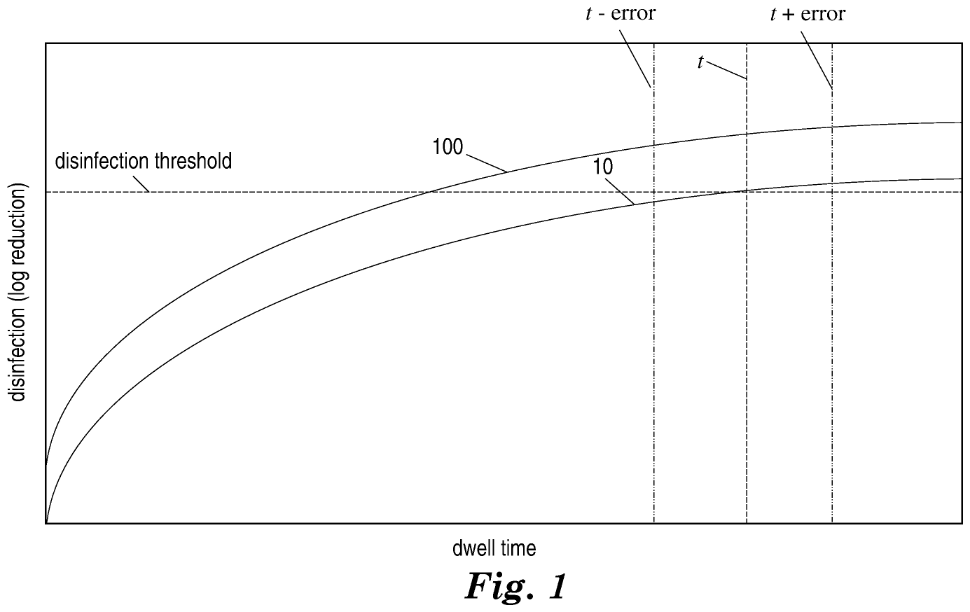

FIG. 1 is a graph showing the relationship between disinfection and dwell time for both an embodiment of a method of removing pathogens from a surface described herein and for a conventional method. FIG. 1 is intended to be a generalized example of typical curves of disinfection versus dwell time. For this reason, both axes are unitless. Conventional curve 10 shows the disinfection (as a log reduction) for a particular disinfectant increasing as a function of dwell time. Conventional curve 10 reaches the disinfection threshold at time t. It may also be appreciated that conventional curve 10 is below the disinfection threshold for a time even slightly shorter than t. The disinfection threshold may vary based on the jurisdiction and type of product: for example, a 6-log reduction (a millionfold or 99.9999% reduction) is commonly used as a desired threshold for commercial disinfectants. Other thresholds may be associated with a particular disinfectant or with a particular environment, such as 4-log, 5-log, or even greater than 6-log.

Curve 100 represents the method described herein. In providing a wipe including a guanidinyl-containing polymer coating. While the curve is generally the same shape a conventional curve 100, the initial removal of pathogens from the surface means that curve 100 is above the disinfection threshold for a range of times around the associated dwell time, t. Depending on the particular application, and the particular disinfectant, the error associated with the actual dwell time t may be ±40% of the associated disinfection time for a particular pathogen. In some embodiments, the associated error may be ±30%. In some embodiments, the associated error may be ±20%. In some embodiments, the associated error may be ±10%.

The associated disinfection time for the disinfectant is typically a registered value, based on testing by one or more standard methods. For example, the associated disinfection time may have been determined (or may be determined) by ASTM E1153-14. In some embodiments, the associated disinfection time may have been determined (or may be determined) by AOAC 961.02.

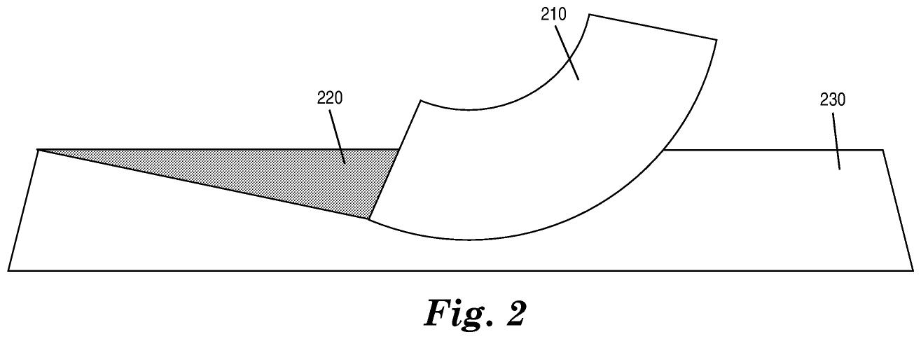

FIG. 2 is a top perspective schematic of contacting a surface with a wipe carrying a liquid disinfectant. Wipe 210 including a guanidinyl-containing polymer coating is contacted to surface 230. Liquid disinfectant 220 is left in contact with surface 230.

Wipe 210 may be any suitable wipe or cloth. In some embodiments, wipe 210 is a disposable wipe. Disposable wipes have certain advantages—some viable number of microbes are often left on the wipe even using thorough laundering procedures. Therefore, disposable wipes eliminate the risk of cross-contaminations between surfaces, rooms, or cleaning sessions.

In some embodiments, the wipe includes a nonwoven substrate or fabric. Examples of suitable nonwoven fabrics include, but are not limited to, melt-blown fabrics, spun-bond fabrics, carded fabrics, wetlaid fabrics, and air-laid fabrics. In some embodiments, the wipe includes a woven fabric substrate. In some embodiments, the wipe includes a sponge or cellulosic substrate. The substrate may include organic polymeric materials, including poly(meth)acrylates, poly(meth)acrylamides, polyolefins, poly(isoprenes), poly(butadienes), fluorinated polymers, chlorinated polymers, polyamides, polyimides, polyethers, poly(ether sulfones), poly(sulfones), poly(vinyl acetates), copolymers of vinyl acetate, such as poly(ethylene)-co-poly(vinyl alcohol), poly(phosphazenes), poly(vinyl esters), poly(vinyl ethers), poly(vinyl alcohols), poly(carbonates), polyurethanes, and cellulosic materials. Suitable polyolefins include, but are not limited to, poly(ethylene), poly(propylene), poly(l-butene), copolymers of ethylene and propylene, alpha olefin copolymers (such as copolymers of ethylene or propylene with 1-butene, 1-hexene, 1-octene, and 1-decene), poly(ethylene-co-1-butene) and poly(ethylene-co-1-butene-co-1-hexene). Suitable fluorinated polymers include, but are not limited to, poly(vinyl fluoride), poly(vinylidene fluoride), copolymers of vinylidene fluoride (such as poly(vinylidene fluoride-co-hexafluoropropylene), and copolymers of chlorotrifluoroethylene (such as poly(ethylene-co-chlorotrifluoroethylene). Suitable polyamides include, but are not limited to, poly(iminoadipoyliminohexamethylene), poly(iminoadipoyliminodecamethylene), and polycaprolactam. Suitable polyimides include, but are not limited to, poly(pyromellitimide). Suitable poly(ether sulfones) include, but are not limited to, poly(diphenylether sulfone) and poly(diphenylsulfone-co-diphenylene oxide sulfone). Suitable copolymers of vinyl acetate include, but are not limited to, poly(ethylene-co-vinyl acetate) and such copolymers in which at least some of the acetate groups have been hydrolyzed to afford various poly(vinyl alcohols). Suitable cellulosic materials include cotton, rayon, and blends thereof. In some embodiments, the substrate is formed from propylene polymers (e.g., homopolymer or copolymers). Polypropylene polymers, particularly polypropylene homopolymers, can be desirable for some applications due to properties such as non-toxicity, inertness, low cost, and the ease with which it can be extruded, molded, and formed into articles. Polypropylene polymers can be formed, for example, into porous sheets of woven or nonwoven fibers.

In some embodiments, wipe 210 is in individual, single-use sized sheets that correspond to a comfortable size to manipulate by hand. In some embodiments, wipe 210 is provided in a continuous or extended roll that may be cut or separated into sheets of desired length. In some embodiments, wipe 210 is an extended roll that includes perforations or other easy-tear features that provide a straightforward way to separate wipes into a useable portion when ready to use. Wipe 210 may be any suitable shape, size, and thickness.

Wipe 210 includes a guanidinyl-containing polymer coating. The guanidinyl-containing polymer coating may be a cationic polymer coating. The guanidinyl group can be located at any position in the polymer. In most embodiments, the guanidinyl group is part of a pendant group attached to the backbone of the polymer. In some embodiments, however, the guanidinyl group is part of backbone of the polymer. As used herein, the term “guanidinyl” refers to a group of the formula —NR³—C(═NR⁴)—NR⁴R⁵. If the guanidinyl group is part of a pendant group, the group R³ refers to hydrogen, C₁-C₁₂ (hetero)alkyl, or C₅-C₁₂ (hetero)aryl. If the guanidinyl group is part of the backbone of the polymer, the group R³ can refer to a residue of a polymer chain. Each group R⁴ is independently hydrogen, C₁-C₁₂ (hetero)alkyl, or C₅-C₁₂ (hetero)aryl. Group R⁵ is hydrogen, C₁-C₁₂ (hetero)alkyl, C₅-C₁₂ (hetero)aryl, or a group of formula —N(R⁴)₂. The guanidinyl group can be part of a biguanidinyl group that is of formula —NR³—C(═NR⁴)—NR⁴—C(═NR⁴)—NR⁴R⁵ where the groups R³, R⁴, and R⁵ are the same as defined above. Guanidinyl-containing polymers are described in U.S. Pat. No. 10,087,405 (Swanson et al.), which is hereby incorporated by reference in its entirety.

A liquid disinfectant 220 is carried by the wipe. Suitable disinfectants include lower alcohols, oxidizing agents (e.g., hydrogen peroxide, peracetic acid, sodium hypochlorite, and the like), phenolics, and quaternary ammonium compounds. Such disinfectants have an associated dwell time required to provide a desired level of disinfection for at least one pathogen (e.g., 6-log reduction), as measured or determined by test methods such as ASTM E1153-14 or AOAC 961.02. For example, a typical dwell time associated with a quaternary ammonium disinfectant (a “quat” disinfectant) may be 10 minutes. The liquid disinfectant may be provided in a ready-to-use format or as a concentrate diluted by water before use. In some embodiments, the liquid disinfectant is added to the wipe at the time of application. For example, the liquid disinfectant may be provided in a spray or squirt dispenser and added at the time of application. In some embodiments, the wipe is impregnated with the liquid disinfectant prior to the time of application. For example, the wipes can be packaged pre-wet with disinfectant or can be provided in a bucket that accepts liquid disinfectant, so that wipes are dispensed impregnated with the liquid disinfectant.

Surface 230 may be any suitable surface. Surface 230 may be a hard, nonporous surface such as a countertop, door, wall, or floor. Other surfaces may be appropriate, depending on the disinfectant, such as porous surfaces or absorbent surfaces such as carpet, upholstery, or bedding.

Wipe 210 leaves liquid disinfectant 220 on surface 230 when applies, and is left to dwell between t−error and t+error, where t is the disinfection time associated with the particular liquid disinfectant for at least one pathogen, and the error is the associated error—in some embodiments, up to ±40%.

Descriptions for elements in figures should be understood to apply equally to corresponding elements in other figures, unless indicated otherwise. The present invention should not be considered limited to the particular embodiments described above, as such embodiments are described in detail in order to facilitate explanation of various aspects of the invention. Rather, the present invention should be understood to cover all aspects of the invention, including various modifications, equivalent processes, and alternative devices falling within the scope of the invention as defined by the appended claims and their equivalents.

### EXAMPLES

Objects and advantages of this disclosure are further illustrated by the following examples, but the particular materials and amounts thereof recited in these examples, as well as other conditions and details, should not be construed to unduly limit this disclosure.

Unless otherwise noted, all parts, percentages, ratios, etc. in the examples and the rest of the specification are by weight, and all reagents used in the examples were obtained, or are available, from general chemical suppliers such as, for example, Sigma-Aldrich, St. Louis, MO, or may be synthesized by conventional methods. The following abbreviations are used in this section: L=liter, mL=milliliter, min=minutes, hr=hours, g=gram, “=inch, cm=centimeter, ° C.=degrees Celcius, gsm=grams per square meter, MW=molecular weight, rpm=revolutions per minute. Unless otherwise noted, all parts, percentages, ratios, etc. in the Examples and the rest of the specification are by weight.

### Materials

| Abbreviation | Description and Source |
|---|---|
| milliQ water | Deionized water, filtered 18 megaohm |
| | water from a MilliQ Synthesis A10 system, |
| | obtained from Millipore, Waltham, MA |
| BUDGE | 1,4-butanediol diglycidyl ether, obtained |
| | from TCI America, Portland, OR |
| G-PEI | Guanylated polyethylenimine (described |
| | in Preparatory Example 1) |
| Sontara 8004 | Nonwoven sheet (PET, 54 gsm), |
| | obtained from Jacob |
| | Holm Group, Basel, Switzerland |

### Test Method

Test Method for Removal of Microorganisms from a Microorganism-Contaminated Surface

The test method was performed as described in U.S. Pat. No. 10,087,405 (Swanson et al.) with the following modifications:

Staphylococcus aureus ATCC 6538, instead of Clostridium sporogenes ATCC 3584 spores, was cultured overnight for at least 20 hr in Tryptic Spy Broth (Becton, Dickinson and Company, Franklin Lakes, NJ) media while shaking at 200 rpm and a temperature of 37° C. The overnight culture (5 mL) was centrifuged at 14,000 rpm for 1 min. The supernatant was pipetted off without disturbing the pellet and replaced with 5 mL of Butterfield's buffer (3M Co., St Paul, MN). Centrifugation and buffered change were repeated two more times (3 total rinses with Butterfield's buffer). On the final rinse step, pipetted off buffer and replaced with Butterfield's buffer (5 mL) containing 25% fetal bovine serum (Gibco, Thermo Fisher Scientific, Waltham, MA) as a soil load. 100 microliters of bacterial stock were inoculated and dried on each surface prior to wiping.

Disinfectant solutions were added to nonwovens, or wipes, in a plastic bag at loading weights of 2.0 to 3.5 times the weight of the wipe. For example, 2 times loading was 2 grams of solution per gram wipe. The disinfectant solution was spread throughout the nonwovens in the plastic bag using a hand roller.

Neutralizing broth used for these experiments was Dey-Engley neutralizing broth (Becton, Dickinson and Company, Franklin Lakes, NJ).

Since bacterial spores were not used in these experiments, no heat shock treatment was performed.

A dilution series from 1:10 to 1:100,000,000 was prepared using sterile Butterfield's buffer (9 mL flip top tubes, available from 3M Co., St. Paul, MN).

### Preparatory Example 1. 25% Guanylated Polyethylenimine (25% G-PEI)

Polyethylenimine, 750,000 MW (2037.1 grams of a 33% w/w solution in water, BASF Lupasol PS) and distilled water (˜1000 g) was charged to a 12 L vessel equipped with overhead stirring. O-methylisourea hemisulfate (481.1 grams, Alfa Aesar) was added to the vessel and enough deionized water was added to bring the total weight to approximately 4017 grams. The reaction mixture was allowed to stir at ambient temperature overnight (about 22 hours). Analysis by NMR spectroscopy indicated conversion to the desired product having 25% of the amine groups of polyethylenimine (primarily the primary amine groups) converted to guanidines. Percent solids was determined to be 24.6% using an Ohaus moisture balance (model number MB35, obtained from the Ohaus Corporation, Parsippany, NJ). The pH of the solution was not adjusted.

### Example 1. 25% G-PEI Coated Nonwoven Crosslinked with BUDGE

Guanylated-polyethylenimine (GPEI; 40.65 grams of 24.6% by weight solids) was diluted with distilled water up to 250 grams (Solution A) and butanediol diglycidyl ether (4.65 grams; TCI Lot URWDG-DH) was diluted in distilled water up to 250 grams (Solution B). Solution A and B were mixed together by hand vigorously. The resulting mixture contained 2% G-PEI and enough crosslinker to react with 20% of available amines. The coating solution (15 mL) was added to a plastic bag containing a nonwoven sheet (10″×12″ (25.4 cm×30.5 cm) sheets; SONTARA 8004, 54 gsm, 100% PET). The plastic bag was closed, and the solution was pushed through the nonwoven using a roller. The wet nonwoven was transferred to an aluminum pan and dried for 20 min at 110° C. Weight gains were determined before and after coating. A total of 30 nonwovens sheets were coated.

### Comparative Example 1. Uncoated Wipe

Sontara 8004 nonwoven not coated with G-PEI and BUDGE mixture.

### Example 2. Quaternary Ammonium Compound Containing Disinfectant

3M HB Quat Disinfectant Cleaner Concentrate 25A (3M Co., St. Paul, MN) was diluted with distilled water at a concentration dilution ratio of 1:365, according to manufacturer's instructions. For Staphylococcus aureus, 3M HB Quat Disinfectant Cleaner Concentrate 25A has an associated 6-log disinfection time of 10 minutes. Nonwovens from Example 1 and Comparative Example 1 were loaded at 2.0 to 3.5 times the weight of the wipe. The recovery control was 8.30 log with a 1.0 log limit of detection. The time it took for the surface to dry after wiping and log reduction for each individual wipe tested are reported in Table 1.

TABLE 1

| | HB Quat Disinfectant | | Log |
|---|---|---|---|
| | Loading | Dry | Reduction |
| | (g solution per g | Time | of |
| Sample | nonwoven) | (min) | S. aureus |
| Example 1 | 2X | 0.42 | 7.3 |
| Example 1 | 2X | 1.53 | 6.3 |
| Example 1 | 2.5X | 1.46 | 6.6 |
| Example 1 | 2.5X | 6.21 | 6.8 |
| Example 1 | 2.5X | 4.17 | 5.8 |
| Example 1 | 3.0X | 11.25 | 7.3 |
| Example 1 | 3.0X | 13.05 | 7.3 |
| Example 1 | 3.0X | 4.41 | 7.3 |
| Example 1 | 3.5X | 18.13 | 7.3 |
| Example 1 | 3.5X | 21.16 | 7.3 |
| Example 1 | 3.5X | 10.26 | 7.3 |
| Comparative Example 1 | 2X | 3.31 | 4.6 |
| Comparative Example 1 | 2X | 2.11 | 5.3 |
| Comparative Example 1 | 2.5X | 7.03 | 4.2 |
| Comparative Example 1 | 2.5X | 5.37 | 4.7 |
| Comparative Example 1 | 3.0X | 5.58 | 5.0 |
| Comparative Example 1 | 3.0X | 8 | 5.3 |
| Comparative Example 1 | 3.0X | 7.15 | 5.5 |
| Comparative Example 1 | 3.5X | 15.5 | 7.3 |
| Comparative Example 1 | 3.5X | 11.59 | 7.3 |
| Comparative Example 1 | 3.5X | 11.14 | 5.6 |

### Example 3. Thymol-Containing Disinfectant

Benefect Botanical Disinfectant (Benefect Corporation, Ontario, Canada) was obtained in a ready-to-use solutions. For Staphylococcus aureus, Benefect Botanical Disinfectant has a published 5-log disinfection time of 10 minutes. Nonwovens from Example 1 and Comparative Example 1 were loaded at 2.0 to 3.5 times the weight of the wipe. The recovery control was 8.31 log with a 1.0 log limit of detection. The time it took for the surface to dry after wiping and log reduction for each individual wipe tested are reported in Table 2.

TABLE 2

| | Benefect Botanical | | Log |
|---|---|---|---|
| | Disinfectant | Dry | Reduction |
| | (g solution per g | Time | of S. |
| Sample | nonwoven) | (min) | aureus |
| Example 1 | 2.5X | 1.12 | 3.75 |
| Example 1 | 2.5X | 2.10 | 4.90 |
| Example 1 | 2.5X | 1.20 | 4.99 |
| Example 1 | 2.5X | 1.05 | 4.99 |
| Example 1 | 3.0X | 3.26 | 5.32 |
| Example 1 | 3.0X | 5.57 | 5.63 |
| Example 1 | 3.0X | 2.50 | 5.84 |
| Example 1 | 3.0X | 6.02 | 6.41 |
| Example 1 | 3.5X | 11.10 | 7.31 |
| Example 1 | 3.5X | 12.06 | 7.31 |
| Example 1 | 3.5X | 12.17 | 5.82 |
| Example 1 | 3.5X | 10.15 | 5.92 |
| Comparative Example 1 | 2.5X | 3.20 | 4.62 |
| Comparative Example 1 | 2.5X | 3.26 | 4.10 |
| Comparative Example 1 | 2.5X | 1.38 | 3.35 |
| Comparative Example 1 | 2.5X | 2.02 | 3.94 |
| Comparative Example 1 | 3.0X | 4.33 | 4.85 |
| Comparative Example 1 | 3.0X | 7.12 | 4.13 |
| Comparative Example 1 | 3.0X | 5.11 | 3.71 |
| Comparative Example 1 | 3.0X | 6.12 | 4.22 |
| Comparative Example 1 | 3.5X | 7.50 | 3.98 |
| Comparative Example 1 | 3.5X | 6.36 | 5.53 |
| Comparative Example 1 | 3.5X | 8.10 | 7.01 |
| Comparative Example 1 | 3.5X | 8.00 | 5.44 |

What is claimed is:

1. A method of removing pathogens from a surface, comprising:
providing a wipe including a guanidinyl-containing polymer coating;
providing a liquid disinfectant having an associated disinfection time for at least one pathogen;
contacting a surface with the wipe carrying the liquid disinfectant;
allowing the liquid disinfectant to be in contact with the surface for a time, t, until removal or evaporation;
wherein t is the associated disinfection time for the at least one pathogen, with an associated error;
wherein the associated error is ±40% of the associated disinfection time for the at least one pathogen.

2. The method of claim 1, wherein the associated error is ±30% of the associated disinfection time for the at least one pathogen.

3. The method of claim 1, wherein the associated error is ±20% of the associated disinfection time for the at least one pathogen.

4. The method of claim 1, wherein the associated error is ±10% of the associated disinfection time for the at least one pathogen.

5. The method of claim 1, wherein the liquid disinfectant is added to the wipe at the time of application.

6. The method of claim 1, wherein the wipe is impregnated with the liquid disinfectant prior to application.

7. The method of claim 1, wherein the liquid disinfectant is sprayed or applied to the surface before contacting the surface with the wipe.

8. The method of claim 1, wherein the associated disinfection time for the at least one pathogen is determined by ASTM E1153-14.

9. The method of claim 1, wherein the associated disinfection time for the at least one pathogen is determined by AOAC 961.02.

10. The method of claim 1, wherein the associated disinfection time for at least one pathogen is a time registered with a governmental regulatory entity.

11. The method of claim 1, wherein the associated disinfection time for the at least one pathogen is a 6-log disinfection time.

12. The method of claim 1, wherein the associated disinfection time for the at least one pathogen is a 4-log disinfection time.

13. The method of claim 1, wherein the surface includes a non-horizontal surface.

14. The method of claim 1, wherein the surface includes a non-planar surface.

15. The method of claim 1, wherein the guanidinyl-containing polymer coating is a cationic coating.

16. The method of claim 1, wherein the wipe comprises a non-woven fabric.

17. The method of claim 1, wherein the liquid disinfectant has a second associated disinfection time for at least one other pathogen; wherein an associated disinfection time for the at least one other pathogen is less than t.

18. The method of claim 1, wherein the liquid disinfectant is a quaternary ammonium disinfectant.
